(19) United States
(12) Patent Application Publication
(10) Pub. No.: US 2014/0024720 A1
(43) Pub. Date: Jan. 23, 2014

(54) PHARMACEUTICAL COMPOSITION CONTAINING CYCLOBENZAPRINE SUITABLE TO INTRANASAL ADMINISTRATION

(71) Applicant: Massimiliano Borsa, Vimodrone(Mi) (IT)

(72) Inventor: Massimiliano Borsa, Vimodrone(Mi) (IT)

(73) Assignee: Campiglo Consulting SRL, Milano (IT)

(21) Appl. No.: 14/110,046

(22) PCT Filed: Apr. 2, 2012
(86) PCT No.: PCT/IB12/00657

§ 371 Date: Oct. 4, 2013

(30) Foreign Application Priority Data
Apr. 6, 2011 (IT) MI2011A000558

(51) Int. Cl. A61K 9/00

(52) U.S. Cl. CPC A61K 9/0043
USPC 514/654

(57) ABSTRACT

A pharmaceutical composition, containing as active principle cyclobenzaprine hydrochloride, 3-(5H-dibenzo[a,d]cyclo-epten-5-yliden)-N,N-dimethyl-1-propanamine hydrochloride, in form suitable to be administered by itranasal route, is described. Said pharmaceutical composition allows a remarkable absorption rapidity of the active principle, does not undergo the first hepatic passage, has an excellent tolerability with low tonicity formulations and does not show any contraindication in the short period administrations.

It is the object of the present invention a pharmaceutical composition containing, as active principle, cyclobenzaprine hydrochloride, 3-(5H-dibenzo[a,d]cyclo-epten-5-yliden)-N,N-dimethyl-1-propanamine hydrochloride, known as central action muscle relaxant agent widely utilized in the therapy to relieve pain from muscular spasms, spasticity or similar clinical alterations. In said pharmaceutical composition cyclobenzaprine is present in form of aqueous solution suitable to be administered by aerosol: by said route it has shown to supply a rapid absorption of the active principle therein contained thus obtaining a rapid answer to the painful state to be defected. Besides the absorption rapidity of the active principle, the pharmaceutical composition of the invention shows the advantage of not undergoing the first hepatic passage, has an excellent tolerability with low tonicity formulations and does not show any contraindication in the short administration period.

### STATE OF THE ART

Cyclobenzaprine hydrochloride, or 3-(5H-dibenzo[a,d] cyclo-epten-5-yliden)-N,N-dimethyl-1-propanamine hydrochloride, is a central action muscle relaxant agent utilized in terapy, in the pharmaceutical form of 5 and 10 mg tablets, in the worldwide most important markets.

Cyclobenzaprine is absorbed per os, metabolized at hepatic level, excreted by renal route, with a plasmatic half time of 18 hours (8-37 hours). The effect of a single administration is shown after one hour from the oral taking and can last up to 12 or more hours; the dosage is 3 administrations/day.

Formulations with modified release are available for one per day administration.

In the art numerous publications are known concerning the use in therapy of cyclobenzaprine: some are referred to the pharmaceutical form of tablets containing up to 25 mg of one of its pharmaceutically acceptable salts, mainly as the hydrochloride, and/or aqueous solution formulations for oral use such as, for instance, drinkable syrups or injectable solutions, with a dosage limit of 25 mg/mL. In other patent texts such as, for instance, GB 1339636; MX200815323A; WO9416703A1, cylobenzaprine is associated to other complementary drugs, such as diazepam, aspirin, diflunisal, and aceclofenac.

More recently, solution formulations of many active principles, among which cyclobenzaprine, were patented, for inhalatory use with the concentration limit equal to 5%, such as in U.S. Pat. No. 7,501,113B2, on the assumption of using the inhalatory administration route as a substitute of the oral one and thus using comparable single dosages.

The patent publication W02004/019905A1 describes solutions for oral use containing cyclobenzaprine up to 50% concentrations, with a bond of a high concentration of an organic solvent and the use of a gaseous propellant to increase the contact surface between the drug and the mucosa. The aim proposed by said composition is to increase the direct absorption of the oral mucosa avoiding the gastric absorption and then the first hepatic passage effect, as it can be deduced from the absorption scheme indicated in said publication on page 33. Actually, the administration in the oral cavity does not eliminate the drug passage in the stomach, because a part of it is dissolved in the saliva and then absorbed through the stomach.

Cyclobenzaprine formulations aimed to increase the onset rapidity of drug action, as it would be hoped for painful muscle spasms caused by muscular-skeletal deseases, are, at present, not known. In this pathology the daily dose can vary from 10 to 60 mg splitted, for not less than 4 days (Martindale—Thirty-six edition, pag. 1895): a formulation with ligher action rapidity could reduce the recourse to anti-inflammatory/analgesic drugs, generally associated, in practice, to the cyclobenzaprine administration and the muscle relaxant doses, with decrease of side effects such as drowsiness and attention fall.

### DETAILED DESCRIPTION OF THE INVENTION

It is the object of the present invention pharmaceutical compositions for intranasal administration consisting of cyclobenzaprine hydrochloride solutions in water with having pH suitable for said administration. Particularly, said cyclobenzaprine pharmaceutical compositions, are characterized by a pH value in the range 6-7.4.

In said range of pH are particularly preferred those pH values comprised between 7,0 and 7,4 to which corresponds an optimized partial presence of the active principle in a not ionized form, therefore able to go through the mucosa.

At a pH higher than 7.4 the cyclobenzaprine aqueous solution shows an opalescence formation due to the cyclobenzaprine base not soluble in the buffer solution: to the purpose, to the pH 7.4 formulation a small quantity of ethanol has been poured in to avoid the formation of said opalescence.

The aqueous solutions containing 5% of cyclobenzaprine hydrochloride are practically isotonic (400 mOsmol/L or 0.88 g-eq di NaCl), while those at 10 and 15% are ipertonic, being the contribution of the buffer solutions nearly negligible.

Combining the concentrations of the indicated range and the possible volume of the pumps available on the market (50-70-100 μL), various administration posologies can be proposed, for instance:
5% aqueous cyclobenzaprine hydrochloride solution with 100 μL pump: one supply corresponds to 5 mg active product;
10% aqueous cyclobenzaprine hydrochloride solution with 50 μL pump: one supply corresponds to 5 mg active product;
15% acqueous cyclobenzaprine hydrochloride solution with 70 μL pump: one supply corresponds to 10.5 mg active product and other at intermediate concentrations.

The final choice is made on the basis of the local tolerability/plasmatic levels ratio.

The compositions object of the invention can also include buffer agents, preservatives, mucoadhesive and absorption enhancer substances. Said substances are of the kind traditionally used in the art and, in case of buffer agents, they are preferably selected among phosphates and acetates; in case of absorption enhancer substances, the preferred ones are chitosan, methylpyrrolidone and sodium cholate; in case of preservatives the preferred ones are selected among benzyl alcohol, quaternary ammonium salts, hydroxybenzoic esters, such as benzalkonium chloride, methyl and propyl parahydroxybenzoate, while the preferred mucoadhesive substance is sodium hyaluronate.

The cyclobenzaprine hydrochloride formulations for intranasal route object of the invention have absorption rapidity, bypass the first hepatic passage and have an excellent tolerability with low tonicity formulations. They do not show any contraindication for the administration of a few days short period.

The following Examples have the purpose of better illustrate the invention without in any case limiting it.

### EXAMPLE 1

Grams 150.00 of cyclobenzaprine hydrochloride, to which 300.00 mL of purified water and 50.00 mL of 95% ethanol were added, are put under magnetic stirring and further added with 2.00 g of benzalkonium chloride. The limpid solution by adding 70.00 mL 1N sodium hydroxide is brought to about the final pH value by means of a pHmeter and, then, added with 500,00 mL of a buffer solution. The pH is adjusted to the desired value by 1N sodium hydroxide and the solution brought to 1.00 L final volume with purified water. The resulting solution is limpid and has a pH that may differ, from the desired value, in the range of ±0.2 units.

Alternatively, the above described process can be carried out substituting the benzalkonium chloride with 10.00 g benzyl alcohol.

Operation is carried out as per the prevoiusly described Example 1, to obtain the below indicated solutions for intranasal use (100 mL).

### EXAMPLES 2-10

### With Benzalkonium Chloride

| | Example 2 | | Example 3 | | Example 4 | |
|---|---|---|---|---|---|---|
| Cyclobenzaprine•HCl | 15.00 | g | 10.00 | g | 5.00 | g |
| Benzalkonium Chloride | 0.20 | g | 0.20 | g | 0.20 | g |
| 95% Ethanol | 5.00 | mL | 5.00 | mL | 5.00 | mL |
| 1N Sodium hydroxide | 7.00 | mL | 5.00 | mL | 3.00 | mL |
| Phosphate buffer | 50.00 | mL | 50.00 | mL | 50.00 | mL |
| pH 7.4 (0.03M) | | | | | | |
| Water to | 100.00 | mL | 100.00 | mL | 100.00 | mL |
| | Example 5 | | Example 6 | | Example 7 | |
| Cyclobenzaprine•HCl | 15.00 | g | 10.00 | g | 5.00 | g |
| Benzalkonium | 0.20 | g | 0.20 | g | 0.20 | g |
| chloride | | | | | | |
| 1N Sodium hydroxide | 1.00 | mL | 0.50 | mL | 0.50 | mL |
| Phosphate buffer | 50.00 | mL | 50.00 | mL | 50.0 | mL |
| pH 6.5 (0.03M) | | | | | | |
| Water to | 100.00 | mL | 100.00 | mL | 100.00 | mL |
| | Example 8 | | Example 9 | | Example 10 | |
| Cyclobenzaprine•HCl | 15.00 | g | 10.00 | g | 5.00 | g |
| Benzalkonium chloride | 0.20 | g | 0.20 | g | 0.20 | g |
| 1N Sodium hydroxide | 4.00 | mL | 3.00 | mL | 2.00 | mL |
| Phosphate buffer pH 7 | 50.00 | mL | 50.00 | mL | 50.00 | mL |
| (0.03M) | | | | | | |
| Water to | 100.00 | mL | 100.00 | mL | 100.00 | mL |

### EXAMPLES 11-13

### With Benzyl Alcohol

| | Example 11 | | Esample 12 | | Example 13 | |
|---|---|---|---|---|---|---|
| Cyclobenzaprine•HCl | 15.00 | g | 10.00 | g | 5.00 | g |
| Benzyl alcohol | 1.00 | g | 1.00 | g | 1.00 | g |
| 95% Ethanol | 5.00 | mL | 5.00 | mL | 5.00 | mL |
| 1N Sodium hydroxide | 7.00 | mL | 5.00 | mL | 3.00 | mL |
| Phosphate buffer | 50.00 | mL | 50.00 | mL | 50.00 | mL |
| pH 7.4 (0.03M) | | | | | | |
| Water to | 100.00 | mL | 100.00 | mL | 100.00 | mL |

### EXAMPLE 14

Grams 150.00 of cyclobenzaprine hydrochloride, added with 300.00 mL purified water and 50.00 mL 95% ethanol, are put under magnetic stirring and added with 2.00 g benzalkonium chloride and 5.00g chitosan. The solution is left under slow stirring for at least six hours, then the limpid solution by adding 70.00 mL sodium hydroxide 1N is adjusted to about the final pH value by means of a pHmeter and then added with 500.00 mL buffer solution. The pH is then adjusted to the desired value with 1N sodium bydroxide and the solution is brought to 1.00 L final volume with purified water. The resulting solution is limpid and has a pH that can differ, from the pH desired value, in the range of ±0.2 units.

Alternatively, the above described process can be carried out replacing the benzalkonium chloride with 10.00 g of benzyl alcohol.

### EXAMPLES 15-17

### With Chitosan

| | Example 15 | | Example 16 | | Example 17 | |
|---|---|---|---|---|---|---|
| Cyclobenzaprine•HCl | 15.00 | g | 10.00 | g | 5.00 | g |
| Benzalkonium chloride | 0.20 | g | 0.20 | g | 0.20 | g |
| 95% Ethanol | 5.00 | mL | 5.00 | mL | 5.00 | mL |
| 1N Sodium hydroxide | 7.00 | mL | 5.00 | mL | 3.00 | mL |
| Chitosan | 0.50 | mL | 0.50 | mL | 0.50 | mL |
| Phosphate buffer | 50.00 | mL | 50.00 | mL | 50.00 | mL |
| pH 7.4 (0.03M) | | | | | | |
| Water to | 100.00 | mL | 100.00 | mL | 100.00 | mL |

### EXAMPLES 18-20

### With Hyaluronic Acid (800-1000 kD)

| | Esempio 18 | | Esempio 19 | | Esempio 20 | |
|---|---|---|---|---|---|---|
| Cyclobenzaprine•HCl | 15.00 | g | 10.00 | g | 5.00 | g |
| Benzalkonium chloride | 0.20 | g | 0.20 | g | 0.20 | g |
| 95% Ethanol | 5.00 | mL | 5.00 | mL | 5.00 | mL |
| 1N Sodium hydioxide | 7.00 | mL | 5.00 | mL | 3.00 | mL |
| Sodium hyaluronate | 0.20 | g | 0.20 | g | 0.20 | g |
| Phosphate buffer pH | 50.00 | mL | 50.00 | mL | 50.00 | mL |
| 7.4 (0.03M) | | | | | | |
| Water to | 100.00 | mL | 100.00 | mL | 100.00 | mL |

In order to evaluate the activity of the composition of the invention administered by intranasal route the following texts have been carried out.

The plasma levels of cyclobenzaprine (CBZ) were determined after a single intranasal dose of 1.5 mg/kg in a volume of 10 μl, and 3.0 mg/kg in a volume of 20 μl, and after a single oral dose of 1.5 mg/kg in New Zealand albino male rabbits.

1. The oral administration was performed with a 0.15% solution, the intranasal administration with a 15% solution.

The composition of the test articles correspond to the following ones.

| | Test article | Test article |
|---|---|---|
| | 0.15% w/v | 15.0% w/v |
| Cyclobenzaprine HCl g | 0.150 | 15.000 |
| Benzalkonium chloride g | 0.200 | 0.200 |
| 0.1N Sodium hydroxyde ml to | pH 7 | pH 7 |
| pH 7 phosphate buffer to ml | 100 | 100 |

2. Six male animals/group were treated with a single dose by intranasal or oral route.

| | | | | | No. of |
|---|---|---|---|---|---|
| Test | | | Volume | Dose | Animals/ |
| Group | Test Item | Route | (μL/kg) | (mg/kg) | Group |
| 1 | Cyclobenzaprine•HCl | oral | 1000 | 1.5 | 6 |
| | (0.15%) | | | | |
| 2 | Cyclobenzaprine•HCl | intranasal | 10 | 1.5 | 6 |
| | (15.0%) | | | | |
| 3 | Cyclobenzaprine•HCl | intranasal | 20 | 3.0 | 6 |
| | (15.0%) | | | | |

3. Fifteen minutes before the drug administration the central artery of the ear was cannulated with a 25-gauge needle catheter and for each animal 7 blood collections at time 0 (TO, before drug administration) and at 15, 30, 60, 90, 120, 180 minutes after drug administration were performed. Three mL of blood sample were collected in heparinized tubes and the volume replaced with a Ringer-Lactate solution. The plasma samples were obtained from blood samples by centrifugation at 3000 g for 5 min. The plasma samples were be stored at −20±2° C. until the LC/MS/MS analysis.

4. Rabbit plasma concentrations of CBZ were determined using a validated LC-MS/MS method in the calibration range of 0.25-300 ng/mL.

Aliquots of 250 μL of rabbit plasma were spiked with 5 μL of internal standard (IS) solution (containing approximately 5000 ng/mL of IS, Imipramine Hydrochloride) in water:methanol (50/50, v/v), after vortex-mixing, 250 μL of borate buffer were added. After vortex-mixing, 3.5 mL of hexane were added. After vortex-mixing for 10 minutes and centrifugation at 10.000 rpm at +4° C. for 10 minutes, aliquots of 3 mL of organic phase were transferred into a single glass tube and dried under vacuum at 40° C. using a Büchi vacuum system. The residues were re-constituted with 100 μL of 20 Mm ammonium acetate buffer solution, 0.1%:acetonitrile (50/50, v/v) solution. The tubes were then capped, vortex-mixed and centrifugated at 4100 rpm at +4° C. for 10 minutes. The final extracts were transferred into an autosampler vial and 4 μL were injected into the LC-MS/MS system.

Chromatographic Condition

| Item | Description | |
|---|---|---|
| Analytical column | Phenomenex, Kinetex, 2.10 × | |
| | 50 mm, 2.6 μm, C18 | |
| Column oven temperature | 25° C. | |
| HPLC Solution (A) | 20 mM Ammonium acetate buffer | |
| | solution pH 3.5, | |
| | 0.1% Formic Acid | |
| HPLC Solution (B) | Acetonitrile | |
| Mobile phase composition | A | B |
| | 63 | 37 |

| | |
|---|---|
| Flow rate | 0.2 mL/min |
| Autosampler temperature | +4° C. |
| Injection volume | 4 μL |
| Autosampler flushing solvent | U.P. water:acetonitrile (50/50, v/v) |
| Retention times | Retention times of cyclobenzaprine |
| | and IS were about 1.86 and 1.85 |
| | minutes respectively |
| Total run time | 8 minutes |

Detection

Positive ion mode using a API/ESI interface and Multiple Reaction Monitoring (MRM).

Mass transitions: 276→149 m/z for cyclobenzaprine
281.1→193 m/z for IS

Calibration curves plot the ratio of the area of the compound and the IS (y) against the analyte concentration (x). A weighted linear regression function (1/x) is used to fit calibration lines and consequently to calculate CBZ concentrations. The lower and upper limits of quantification are 0.25 and 300 ng/mL of plasma samples.

Results

Mean plasma levels (±SD) after treatments are reported (FIG. 1, each data is the mean of six rabbits)

The plasma levels of single animals are closed to the mean profile for each treatment (FIG. 2-4).

The main PK parameters after dose adjusted 3.0 mg/kg values resulted as follows.

| | Oral | Intranasal | Intranasal |
|---|---|---|---|
| | 1.5 mg/kg | 1.5 mg/kg | 3.0 mg/kg |
| Cₘₐₓ (ng/ml) | 1.28 | 69.34 | 65.14 |
| CV % | 8.43 | 1.44 | 11.74 |
| AUC₀₋ₜ (ng_{*}h/ml) | 2.08 | 60.13 | 54.61 |
| CV % | 10.05 | 4.87 | 4.41 |
| AUC_{∞} (ng_{*}h/ml) | 3.44 | 65.82 | 65.61 |
| CV % | 30.69 | 5.59 | 4.23 |
| Tₘₐₓ (h) (median) | 0.50 | 0.50 | 0.50 |
| range | 0.50-0.50 | 0.25-0.50 | 0.50-0.50 |
| λ_{z} (h⁻¹) | 0.361 | 0.940 | 0.452 |
| t_{1/2} (h) | 2.073 | 0.821 | 1.565 |

Cyclobenzaprine is well absorbed by intranasal route and its rate of absorption is higher compared with oral route. Cₘₐₓ and AUC resulted linear with the dose by intranasal route, but much higher than by oral route. The results obtained by intranasal administration of the proposed formulations of cyclobenzaprine hydrochloride show a clear enhancement of its therapeutic performance when compared to those obtained by the oral administration.

1. A pharmaceutical composition comprising:
an aqueous solution comprising 5 to 20% w/v of cyclobenzaprine hydrochloride, wherein the aqueous solution has a pH of 6 to 7.4, and
optionally one or more members selected from the group consisting of pharmaceutically acceptable preservatives, buffer solutions, muco-adhesive and absorption enhancer substances,
wherein the pharmaceutical composition has a muscle relaxant activity and is suitable for spray intranasal administration.

2. The pharmaceutical composition of claim 1, comprising at least one pharmaceutically acceptable preservative selected from the group consisting of benzyl alcohol, an ammonium quaternary salt, and a hydroxybenzoic ester, in a concentration suitable for spray administration.

3. The pharmaceutical composition of claim 2, wherein the preservative is selected from the group consisting of benzyl alcohol, benzalkonium chloride, a methyl-parahydroxybenzoate, and a propyl-parahydroxybenzoate.

4. The pharmaceutical composition of claim 1, comprising at least one absorption enhancer substance selected from the group consisting of chitosan, methylpyrrolidone and sodium cholate.

5. The pharmaceutical composition of claim 1, comprising a phosphate or acetate buffer solution.

6. The pharmaceutical composition of claim 1, comprising a muco-adhesive substance that is sodium hyaluronate.

7. A process of administering the pharmaceutical composition of claim 1, the process comprising spray of administering the composition via a 50-120 μL pump to a patient in need thereof.

8. The pharmaceutical composition of claim 1, comprising a suitable pharmaceutically acceptable preservative.

9. The pharmaceutical composition of claim 1, comprising a buffer solution.

10. The pharmaceutical composition of claim 1, comprising a muco-adhesive substance.

11. The pharmaceutical composition of claim 1, comprising an absorption enhancer substance.

12. The pharmaceutical composition of claim 2, wherein the preservative is benzyl alcohol.

13. The pharmaceutical composition of claim 2, wherein the preservative is benzalkonium chloride.

14. The pharmaceutical composition of claim 2, wherein the preservative is a methyl-parahydroxybenzoate.

15. The pharmaceutical composition of claim 2, wherein the preservative is a propyl-parahydroxybenzoate.

16. The pharmaceutical composition of claim 4, wherein the absorption enhancer substance is chitosan.

17. The pharmaceutical composition of claim 4, wherein the absorption enhancer substance is methylpyrrolidone.

18. The pharmaceutical composition of claim 4, wherein the absorption enhancer substance is sodium cholate.

19. The pharmaceutical composition of claim 1, comprising a phosphate buffer solution.

20. The pharmaceutical composition of claim 1, comprising an acetate buffer solution.
